(19) United States
(12) Patent Application Publication
(10) Pub. No.: US 2013/0277322 A1
(43) Pub. Date: Oct. 24, 2013

(54) SAFETY AMPOULE HOLDER

(71) Applicant: Osvaldo Antonio TIBERIO, Olivos (AR)

(72) Inventor: Osvaldo Antonio TIBERIO, Olivos (AR)

(21) Appl. No.: 13/451,600

(22) Filed: Apr. 20, 2012

(51) Int. Cl. A61B 19/00

(52) U.S. Cl. 211/74

(57) ABSTRACT

This invention is a plastic device to hold glass ampoules, vials and other containers, designed to prevent sharp injuries when loading them, for use by health workers, in anywhere where they use glass ampoules, in accordance with international biosafety standards. This holder consists of a plastic structure of three parts. The rear part is flat. The base has plastic tabs that help ensure the stability of the ampoules. The top has holes designed to facilitate placement of the ampoules, with a slightly higher diameter than that ampoules will accommodate. This device should be used as follows: Place the ampoule in the corresponding hole, open the ampoules using openers available, load the syringe, rotate 180 degrees the holder on the sharps container so that the ampoule fall by self-gravity, avoiding any contact between the healthcare worker with the broken ampoule, eliminating the possibility of injury.

### BACKGROUND OF THE INVENTION

In recent years the standards of biosecurity and protection of health workers were becoming more accurate in their benefit. This invention was developed for use in any area where it is usual to load syringes with medications that comes in glass ampoules, vials or others container devices, such as nursing rooms, intensive care units, anesthesiology tables and any other places where this type of labor is performed. Ampoule holder refers to a plastic support for medicinal glass ampoules, vials or others container of medical and biological devices, with the aim of reducing accidents for bad breakage and overrides the risk of puncture that occurs when loading the syringe. At present, to open the glass ampoule, are several openers to minimize the risk of injury to the operator (doctor, nurse, etc.). But what is even more dangerous and goes against basic biosecurity practices, is to load the syringes holding the ampoule with no skilled hand and trying to insert the needle into the hole of it, with the clear risk of needlestick to the operator. Another practice for loading syringes (common in anesthetists) is when an assistant offers the opened ampoule and the doctor tries to hit the hole with the risk of hurting the person who is holding the ampoule. These risks are compounded when the health workers need to break large number of ampoules to place the medication in the syringes. Currently there are a lot of regulations in USA, Canada and European Union between others. We are intended to prevent accidents with puncture-proof materials since they have noticed that increased a lot in the last years. In September 1998 California became the first state in the country to enact a law aimed at protecting health care workers. The bill was introduced after a series of articles appeared in the San Francisco Chronicle asserting that one million health care workers are injured in the United States each year as a result of needlestick. Since that time 20 additional states have passed Needlestick Safety Legislation. These states include: Tennessee, Maryland, Texas, New Jersey, West Virginia, Minnesota, Maine, Georgia, Iowa, New Hampshire, Alaska, Connecticut, Oklahoma, Ohio, Massachusetts, New York, Arkansas, Missouri, Rhode Island, and Pennsylvania. Additional Needlestick safety bills have been introduced in several other states. In November 2000, President Clinton signed into law the Needlestick Safety and Prevention Act (HR 5178.) The Department of Labor of the United States has standard regulations under number 1910.1030 whose scope and application is given to protect all workers from possible cuts and punctures with blood or other potentially infectious materials. Canada's occupational safety and health programs are organized and administered at the provincial level; accordingly, regulations relevant to sharps safety and needlestick prevention have been promulgated by provincial authorities. Those provinces that have passed some type of needle safety legislation as of March 2009 are: —Alberta: The Alberta government promulgated a provincial-level Occupational Health and Safety Code (OHS Code) in November 2003 which set standards for protecting the health and safety of workers —British Columbia: In 2007, British Columbia's WorkSafe BC, the provincial body governing occupational safety, amended the Occupational Health and Safety Regulation [Guidelines Part 6—Biological Agents; 6.33-6.40] to require safety-engineered needles for any procedures involving use of hollow-bore needles (e.g., withdrawing blood or body fluids, accessing a vein or artery, administering medications or fluids). This requirement applies to all workplaces, including physician's offices and clinics, patient treatments at their homes, and long-term care facilities, in addition to hospitals). —Manitoba: The Manitoba government passed Bill 23, the Workplace Safety and Health Amendment Act (Needles in Medical Workplaces), on Jun. 9, 2005 (S.M. 2005, c. 15; 3rd Session, 38th Legislature). The law requires that all healthcare workplaces protect workers by implementing safety-engineered needles whenever feasible. Safe work practices in relation to needle use are also required. Another States with biosafety regulations are: —Nova Scotia: The Safer Needles in Healthcare Workplaces Act, passed in 2006, came into effect in January 2007. Under this act, healthcare facilities in Nova Scotia are required to implement safety-engineered needles, with certain exceptions, and to provide instruction and training in their use. Compliance was required no later than one year after the law took effect. —Ontario: The Ontario Regulation 474/07 Needle Safety under the Ontario Occupational Health and Safety Act, the regulation requires hospitals to use safety-engineered needles and needleless devices to replace conventional hollow-bore needles by Sep. 1, 2008. Saskatchewan: Needle Safe Devices and Improved Exposure Control Plans (Document ID 12283)-Saskatchewan's Occupational Health and Safety Act was revised on Oct. 19, 2005, to mandate the use of safer needles in health care and correctional facilities. The revised regulations require employers or contractors to develop and implement an exposure control plan to eliminate or minimize worker exposure if workers are required to handle, use or produce an infectious material or organism or are likely to be exposed at a place of employment. —European Union: Meanwhile, the council of the European Union has implemented under number 2010/32 a Framework Agreement on prevention from sharp injuries in the hospital and healthcare sector concluded by HOSPEEM and EPSU on 17 Jul. 2009. Also, many European countries individually have implemented regulations regarding biosecurity as Germany, France, Spain and the Unite Kingdom, Asian countries like Hong Kong (China), Japan and Australia among others.

### BRIEF SUMMARY OF THE INVENTION

The invention consists of a plastic holder for medicinal glass ampoules, in which after opening, with the different types of ampoules openers available, following the instructions given by the manufacturers of these, will place through the open ampoule base in the hole to suit. Then you proceed to load the contents of the ampoule/ampoules, meeting the standards of good practices. Then you take the holder and it will rotate 180 above the sharps container for them to fall into the same by gravity, avoiding a second chance of accidents by not handling the broken ampoule. This new practice not requires two person for fill syringes doing de work more efficient, or a person trying to hit the hole witch the risk that this practice have. Achieve implement it as a habit at work to protect the health staff, and incorporate it into the Occupational Health and Safety standards. This safety holder systematically eliminates the risk of punctures or cuts caused by needles or broken glass ampoules.

### BRIEF DESCRIPTION OF THE SEVERAL VIEWS OF THE DRAWINGS

FIG. 1 is a front view of ¾ of the device, incorporating some ampoules of different sizes to be understood the functionality of the safety holder. Is Shown in the top holes that have a special design with conical shape to facilitate placement of the ampoule in the safety holder, with diameters slightly greater than the ampoule, which together with the base supports, give stability to the ampoule or vial to charge its content. These holes are designed to 1 milliliter, (ml), 2 ml. (Sharing these two measurements the same holes), 3 ml. and 5 ml, (Sharing these two measurements the same holes) 10 ml. and 20 ml. Is also a show the incline of 25° to the horizontal so that the blisters provide the filling hole with this angle, for the convenience of the health worker.

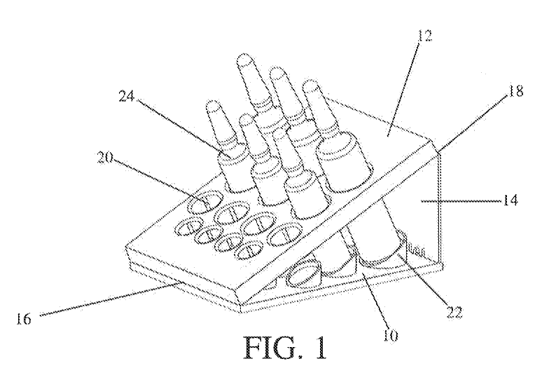

FIG. 2 shows the same in its front view,

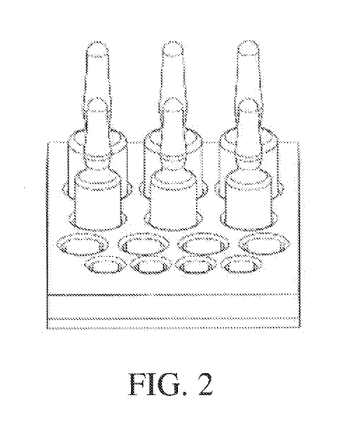

FIG. 3 shows the top view.

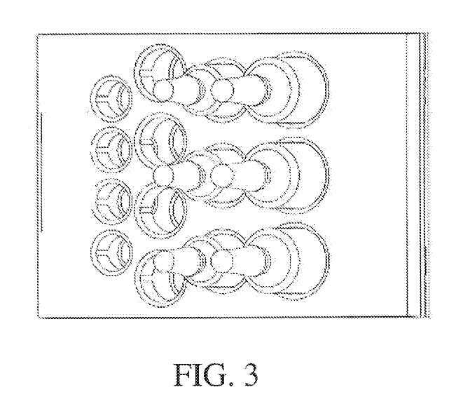

FIG. 4 Side view, in this case with some ampoules to facilitate understanding of the safety holder. The piece, on leaving the matrix, is flat, with two hinges, one between the rear and top (that is perforated portion where the blisters are arranged) and another between the top and base. The base and back are coupled with two hooks which are shown in FIGS. 1, 5, 6, 7.8 and 9.

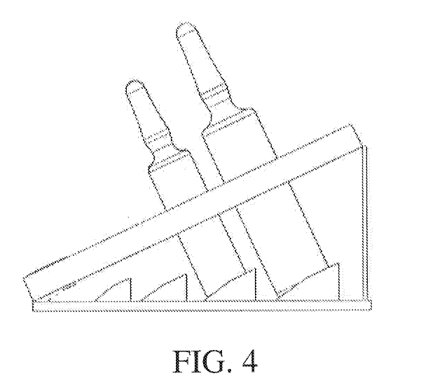

FIG. 5 shows the safety holder as exits from the matrix, showing its internal part. On the left of the figure shows the inside of the base showing where they will support the bases of the ampoule to ensure the stability thereof. Also shows the central part, where the plastic edges are divided into three parts, with the taper downwardly to ensure the stability of the ampoule. Finally on the right side shows the back, which has hooks that are applied to the left for field assembly.

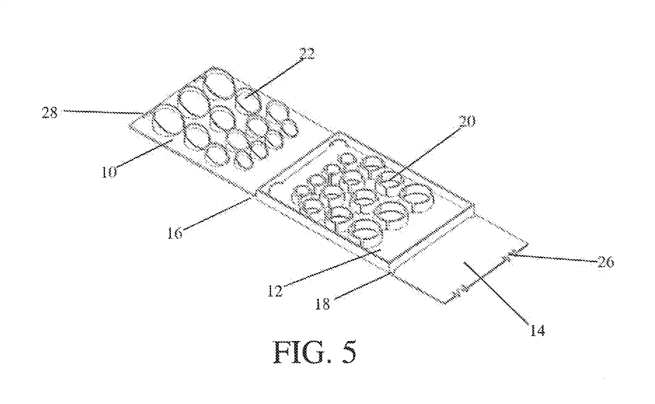

FIG. 6 shows the safety holder displayed by their outer sides, rotated 180 degrees from the previous figure, appreciating the same sectors.

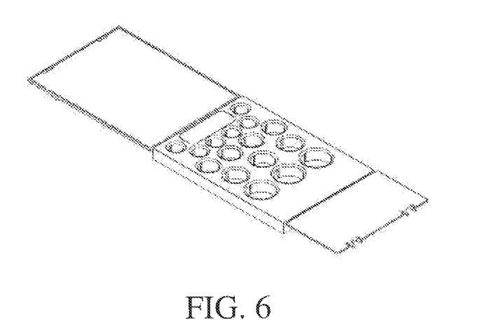

FIG. 7 displays as it exits from the matrix. This figure is shown to appreciate the plastic hinges between the three sectors of the safety holder.

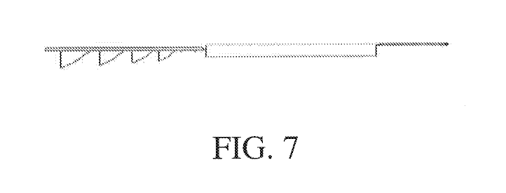

FIG. 8 shows a top view of the safety holder as is deployed out of the matrix on its inner side. This view shows more clearly the hinges between the three sectors of the safety holder, while offering a new view of the hooks used for assembly.

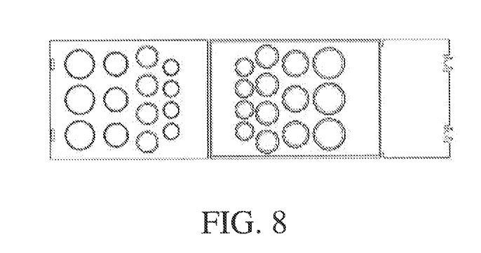

FIG. 9 is a view of the external faces where appreciated as in the previous figure.

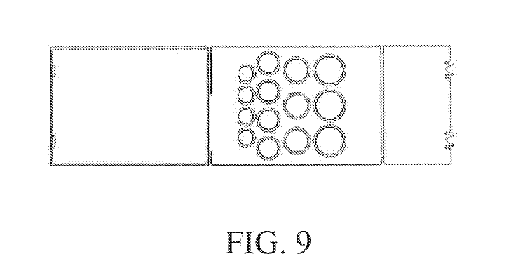

FIG. 10 shows another possibility to use the safety holder, changing the loading angle, having the operator another choice to work for his comfort.

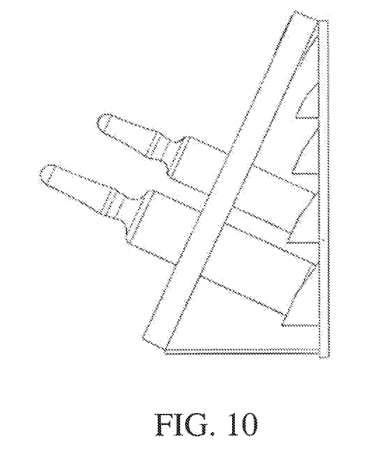

### DETAILED DESCRIPTION OF THE INVENTION

This safety ampoule holder consists of a plastic structure of three parts joined together. One of them is flat, that is the rear. The base has plastic tabs that help ensure the stability of the ampoules. The top has holes designed to facilitate placement of the ampoules in the safety holder, with a diameter slightly higher than that ampoules will accommodate. In this embodiment the angle of the ampoule from the horizontal is approximately 65 degrees. Eventually be used as the basis the rear part, to user's choice, having in this case the ampoule an angle of 25 degrees approximately from the horizontal. At this point of exposure is clear that there will not be a single design of the safety holder, but several. The designs to come may be specific to adapt to the place of use (nursing, intensive care unit, operating room, anesthesia table, etc.), maintaining in all cases the same concept, which is to prevent needlestick loading the syringes from glass ampoules, vials or similar containers. Therefore the process to load the syringes using this safety holder is: Place the ampoule in the safety holder, with the base downwards and neck thereof upward, then place the ampoule opener (existing) on the neck of the ampoule, break the neck of the ampoule using the ampoule opener, following the instructions given from the ampoule opener manufacturer, load the syringe or syringes from the glass ampoule, discard the ampoule taking the safety holder 180 degrees and turning on, dropping the ampoule by gravity avoiding the broken blisters by hands on the usual sharp containers (existing). As shown, the absence of direct contact between the ampoule and the hands of the health worker when loading syringes from glass ampoules, removes the risk of injury or accidental needlestick.

1. (canceled)

2. (canceled)

3. A container holding device comprising:
a first panel, a second panel, and a third panel connected together by a first hinge and a second hinge;
wherein the second panel comprises at least one slot configured to receive a container;
wherein the container holding device is configured to be situated in a standing position and wherein the standing position comprises the second panel folded over the first panel and the second panel supported by the third panel.

4. The container holding device of claim 1, wherein the third panel supports the second panel at an angle relative to the first panel.

5. The container holding device of claim 1, wherein the at least one slot is a plurality of slots.

6. The container holding device of claim 5, wherein the plurality of slots have different sized diameters.

7. The container holding device of claim 1, wherein the first panel further comprises at least one sheath aligned with the at least one slot and wherein the sheath is configured to house at least a portion of the container.

8. The container holding device of claim 1, wherein the container is an ampoule.

9. The container holding device of claim 1, wherein the first panel is connected to the second panel by the first hinge and the second panel is connected to the third panel by the second hinge.

10. The container holding device of claim 9, wherein the first panel comprises a slit and the third panel comprises a protrusion, wherein in the standing position the protrusion fits within the slit.

11. A container holding device comprising:
a base panel comprising at least one sheath; and
a face panel connected to the base panel at a connection point and comprising at least one slot configured to receive an ampoule, wherein the at least one slot is aligned with the at least one sheath,
wherein the face panel is positioned at an acute angle relative to the base panel.

12. The container holding device of claim 11, further comprising a support panel connected to the base panel and the face panel.

13. The container holding device of claim 11, wherein the acute angle is in the range of about 25 degrees to about 65 degrees.
